(19) United States
(12) Patent Application Publication
(10) Pub. No.: US 2021/0052565 A1
(43) Pub. Date: Feb. 25, 2021

(54) USE OF PANTOPRAZOLE IN PREVENTION AND TREATMENT OF LIVER FIBROSIS

(71) Applicant: Biguang TUO, Zunyi (CN)

(72) Inventors: Biguang TUO, Zunyi (CN); Guorong WEN, Zunyi (CN); Jingguo LI, Zunyi (CN); Jiaxing ZHU, Zunyi (CN); Xuemei LIU, Zunyi (CN); Rui XIE, Zunyi (CN); Xiaoying LONG, Zunyi (CN); Jingyu XU, Zunyi (CN); Hai JIN, Zunyi (CN); Jiaxing AN, Zunyi (CN)

(73) Assignee: Biguang TUO, Zunyi (CN)

(21) Appl. No.: 16/993,790

(22) Filed: Aug. 14, 2020

(30) Foreign Application Priority Data
Aug. 19, 2019 (CN) 201910763742.2

(51) Int. Cl. A61K 31/4439; A61P 1/16

(52) U.S. Cl. CPC A61K 31/4439; A61P 1/16

(57) ABSTRACT

The present invention belongs to the technical field of new use of medicaments, and particularly relates to use of pantoprazole in the prevention and treatment of liver fibrosis. The pantoprazole inhibits the expression of receptor TGFβR1 for the profibrotic cytokine TGFβ (transforming growth factor β), the activation of hepatic stellate cells and the epithelial-mesenchymal transition in liver, thereby inhibiting the occurrence of liver fibrosis.

### CROSS-REFERENCE TO RELATED APPLICATIONS

This application claims priority to Chinese patent application No. 201910763742.2 filed Aug. 19, 2019, the contents of which is herein incorporated by reference in its entirety.

### TECHNICAL FIELD

The present invention belongs to the technical field of new use of medicaments, and particularly relates to use of proton pump inhibitor pantoprazole (PPZ) in the prevention and treatment of liver fibrosis.

### BACKGROUND

Liver fibrosis, the precursor of cirrhosis, is a necessary pathological process for various chronic liver diseases to develop into cirrhosis. According to existing findings, it is generally believed that cirrhosis is an irreversible chronic progressive liver disease, but liver fibrosis is a reversible pathological change. Therefore, inhibiting the occurrence and development of liver fibrosis is the key to preventing the development of chronic liver diseases into cirrhosis, and has important clinical significance for the study on liver fibrosis treatment.

Liver fibrosis is manifested by excessive and abnormal deposition of extracellular matrix components in the liver. Chronic liver damage caused by various factors can lead to the occurrence and development of liver fibrosis, and further to cirrhosis. Liver fibrosis is commonly caused by viral hepatitis, alcoholic or nonalcoholic fatty liver disease, autoimmune hepatitis or the like. Theoretically, the best way to prevent the formation of liver fibrosis is to get rid of harmful factors that damage liver. It is true that effective antiviral treatment can prevent the formation and progression of liver fibrosis, but for most patients with advanced alcoholic or nonalcoholic fatty liver disease and hereditary and autoimmune liver disease, it is difficult to get rid of damage factors. In recent years, people have been devoted to studying the cellular and molecular mechanism of the occurrence of liver fibrosis, and hope to find ways to prevent, stop or reverse the progression of liver fibrosis and improve the liver function. Although many compounds have been found to exhibit anti-liver fibrosis effects in in-vitro experiments and animal models, these compounds have little clinical effect. At present, hormone, ursodeoxycholic acid, statins and the like have been used to treat liver fibrosis clinically, but no satisfactory effect has been achieved in preventing the formation and progression of liver fibrosis. Therefore, it is still urgently needed to find effective medicaments for inhibiting the occurrence and development of liver fibrosis. Liver fibrosis is the result of long-term effects of liver-damaging factors, and the occurrence and development of liver fibrosis is a complex process participated by multiple cells and signaling molecules, involving multiple cellular and molecular mechanisms. Liver fibrosis is characterized by abnormal accumulation of fibrocytes and excessive deposition of extracellular matrix components in the liver, and inflammatory lesions and structural disorders. The activation of hepatic stellate cells (HSC) is considered to play a key role in the initiation and progression of liver fibrosis. Many factors, including inflammatory cytokines and chemokines, transforming growth factor β, vascular-derived growth factor and oxidative stress, are involved in the occurrence and development of liver fibrosis.

PPZ, a proton pump inhibitor that can inhibit gastric acid secretion by inhibiting H+-K+-ATPase in parietal cells, has been widely used in the clinical treatment of gastric acid-related diseases (such as peptic ulcer disease).

However, so far, use of PPZ (proton pump inhibitor) has not been found in medicaments for preventing and treating liver fibrosis.

### SUMMARY

In view of this, the present invention is intended to provide use of pantoprazole in the prevention and treatment of liver fibrosis, and thus provide new use of pantoprazole.

In order to realize the objective of the present invention, the present invention provides the following technical solutions.

The present invention provides use of pantoprazole in the prevention and treatment of liver fibrosis.

Preferably, the pantoprazole prevents and treats liver fibrosis by inhibiting the activation of hepatic stellate cells.

The present invention also provides use of pantoprazole in the prevention and treatment of liver damage.

Preferably, the pantoprazole prevents and treats liver fibrosis by inhibiting the increase of collagen fibers in liver tissue.

Preferably, the pantoprazole prevents and treats liver fibrosis by inhibiting the expression of extracellular matrix proteins.

Preferably, the extracellular matrix protein includes one or more of alpha smooth muscle actin (α-SMA), COL1A1 protein and fibronectin.

Preferably, the pantoprazole prevents and treats liver fibrosis by increasing the expression of epithelial phenotype proteins.

The present invention also provides use of a composition including pantoprazole in the prevention and treatment of liver fibrosis.

The present invention provides use of pantoprazole in the prevention and treatment of liver fibrosis, and the pantoprazole inhibits the expression of receptor TGFβR1 for the profibrotic cytokine TGFβ, the activation of hepatic stellate cells and the epithelial-mesenchymal transition in liver, thereby inhibiting the occurrence of liver fibrosis.

### BRIEF DESCRIPTION OF DRAWINGS

FIG. 1 shows the effect of PPZ (proton pump inhibitor) on the histological changes of liver damage in mouse models of liver fibrosis induced by carbon tetrachloride. The liver histology was examined by HE staining. This figure shows representative images for liver histology HE staining. The liver of normal mice has a normal lobule structure with central veins, and liver cells therein are in the form of cords, without regeneration. After administration of carbon tetrachloride, there is infiltration of inflammatory cells, necrosis of liver cells, disorder of hepatic lobule structure, disorder of liver cell cords, and regenerated liver cells in the liver. The liver damage is alleviated to varying degrees for mice of all PPZ groups. Especially in PPZ groups of 5 mg/kg and 10 mg/kg, normal liver cell cords and hepatic lobules can be seen.

FIG. 2 shows the effect of PPZ (proton pump inhibitor) on the deposition of collagen fibers in liver tissue in mouse models of liver fibrosis induced by carbon tetrachloride, as detected by Masson staining. The upper part shows representative images for Masson staining, and the lower part shows the comparison of Masson staining positive areas (i.e., collagen fiber amounts) of all groups. After administration of carbon tetrachloride (control group), deposition of collagen fibers is significantly increased in liver tissue, and PPZ inhibits the deposition of collagen fibers caused by carbon tetrachloride in a dosage-dependent manner. As compared with the normal group, $$P<0.01; and as compared with the control group, #P>0.05, *P<0.05, and **P<0.01.

FIG. 3 shows the effect of PPZ (proton pump inhibitor) on the deposition of collagen fibers in liver tissue in mouse models of liver fibrosis induced by carbon tetrachloride, as detected by Sirius Red staining. The upper part shows representative images for Sirius Red staining, and the lower part shows the comparison of Sirius Red staining positive areas (i.e., collagen fiber amounts) of all groups. After administration of carbon tetrachloride (control group), deposition of collagen fibers is significantly increased in liver tissue, and PPZ inhibits the deposition of collagen fibers caused by carbon tetrachloride in a dosage-dependent manner. As compared with the normal group, $$P<0.01; and as compared with the control group, #P>0.05, *P<0.05, and **P<0.01.

FIG. 4 shows the effect of PPZ (proton pump inhibitor) on the expression of TGFβ receptor TGFβR1, extracellular matrix proteins α-SMA, COL1A1 and fibronectin, and liver cell epithelial phenotype E-cadherin in liver tissue in mouse models of liver fibrosis induced by carbon tetrachloride. The expression of protein is detected by Western Blot. A in FIG. 4 shows representative Western Blot images, and B to F in FIG. 4 show the comparison of protein expressions for all groups. After administration of carbon tetrachloride (control group), the expressions of TGFβR1, α-SMA, COL1A1 and fibronectin are significantly increased in liver tissue, and the expression of E-cadherin is significantly reduced. PPZ inhibits the expressions of TGFβR1, α-SMA, COL1A1 and fibronectin in a dosage-dependent manner, and increases the expression of E-cadherin. As compared with the normal group, $$P<0.01; and as compared with the control group, *P<0.05 and **P<0.01.

FIG. 5 shows the effect of TGFβ on the activation of hepatic stellate cells LX2 and the effect of PPZ on the expression of hepatic stellate cell matrix protein caused by TGFβ. LX2 cells are treated with TGFβ for 48 h and then treated with different dosages of PPZ for 24 h, and protein expressions are detected by Western Blot. A in FIG. 5 shows representative Western Blot images, and B to D in FIG. 5 show the comparison of protein expressions for all groups. TGFβ treatment obviously causes the increased expressions of α-SMA, COL1A1 and fibronectin in LX2 cells, and PPZ decreases the increased expressions of α-SMA, COL1A1 and fibronectin caused by TGFβ in a dosage-dependent manner. As compared with the group untreated with TGFβ, $$P<0.01; and as compared with the control group treated with TGFβ, #P>0.05, *P<0.05 and **P<0.01.

FIG. 6 shows the effect of PPZ (proton pump inhibitor) on the LX2 cell activation and the cell matrix protein expression caused by TGFβ. LX2 cells are treated with different dosages of PPZ for 30 min and then treated with TGFβ for 24 h, and protein expressions are detected by Western Blot. A in FIG. 6 shows representative Western Blot images, and B to D in FIG. 6 show the comparison of protein expressions for all groups. PPZ inhibits the increased expressions of α-SMA, COL1A1 and fibronectin caused by TGFβ in a dosage-dependent manner. As compared with the group untreated with TGFβ, $$P<0.01; and as compared with the control group treated with TGFβ, #P>0.05, *P<0.05 and **P<0.01.

### DETAILED DESCRIPTION

The present invention provides use of pantoprazole in the prevention and treatment of liver fibrosis. In the present invention, the pantoprazole preferably prevents and treats liver fibrosis by inhibiting the activation of hepatic stellate cells. In the present invention, the pantoprazole preferably prevents and treats liver fibrosis by inhibiting the increase of collagen fibers in liver tissue. In the present invention, the pantoprazole preferably prevents and treats liver fibrosis by inhibiting the expression of extracellular matrix proteins. In the present invention, the extracellular matrix protein preferably includes one or more of α-SMA, COL1A1 protein and fibronectin. In the present invention, the pantoprazole preferably prevents and treats liver fibrosis by increasing the expression of epithelial phenotype proteins. In the present invention, the extracellular matrix protein preferably includes one or more of α-SMA, COL1A1 protein and fibronectin.

The present invention also provides use of pantoprazole in the prevention and treatment of liver damage. In the present invention, the liver damage is preferably caused by carbon tetrachloride.

The present invention also provides use of a composition including pantoprazole in the prevention and treatment of liver fibrosis. In the present invention, the composition preferably includes pantoprazole as the only active substance.

The technical solutions provided by the present invention will be described in detail below with reference to examples, but the examples should not be construed as limiting the claimed scope of the present invention.

### Example 1

Effect of PPZ (Proton Pump Inhibitor) on Carbon Tetrachloride-Induced Liver Fibrosis in Mice

Mice with carbon tetrachloride-induced liver fibrosis are animal models of liver fibrosis commonly used in experimental investigations. In order to investigate the effect of PPZ (proton pump inhibitor) on the occurrence of liver fibrosis, 5 to 6 week-old male C57BL6 mice were adopted for experiment. Liver fibrosis models were established by carbon tetrachloride induction, and then the effects of different dosages of PPZ on the occurrence of liver fibrosis were observed in mice. Carbon tetrachloride was administered at escalating doses for 6 weeks. Carbon tetrachloride was dissolved in olive oil. The first dose was administered intragastrically at 0.875 ml/kg; during the next 1 to 3 weeks, carbon tetrachloride was administered intragastrically at 1.75 ml/kg three times a week; and during the next 4 to 6 weeks, carbon tetrachloride was administered intragastrically at 2.5 ml/kg three times a week. After the first dose of carbon tetrachloride had been administered, the mice were divided into a control group and PPZ (proton pump inhibitor) groups of 1 mg/kg, 2 mg/kg, 5 mg/kg and 10 mg/kg, with each group of 12 mice. PPZ, at dosages of 1 mg/kg, 2 mg/kg, 5 mg/kg and 10 mg/kg, or control solution were intraperitoneally administered to mice once every 12 hours. 6 weeks after administration, mice were sacrificed, and the occurrence of liver damage, the deposition of collagen fibers in liver and the molecular markers for liver fibrosis were examined in mice.

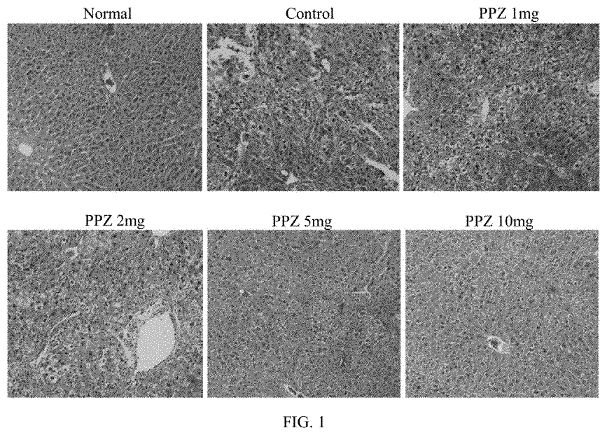

Liver damage in mice was examined by liver histology HE staining. The results showed that the liver in normal mice (given no carbon tetrachloride) had a normal lobule structure with central veins, and liver cells therein were in the form of cords, without regeneration. 6 weeks after administration of carbon tetrachloride, there was infiltration of inflammatory cells, necrosis of liver cells, disorder of hepatic lobule structure, disorder of liver cell cords, and regenerated liver cells in the liver. The liver damage was alleviated to varying degrees for mice of all PPZ groups. Especially in PPZ groups of 5 mg/kg and 10 mg/kg, normal liver cell cords and hepatic lobules could be seen (FIG. 1). It indicates that PPZ can significantly alleviate liver damage caused by carbon tetrachloride.

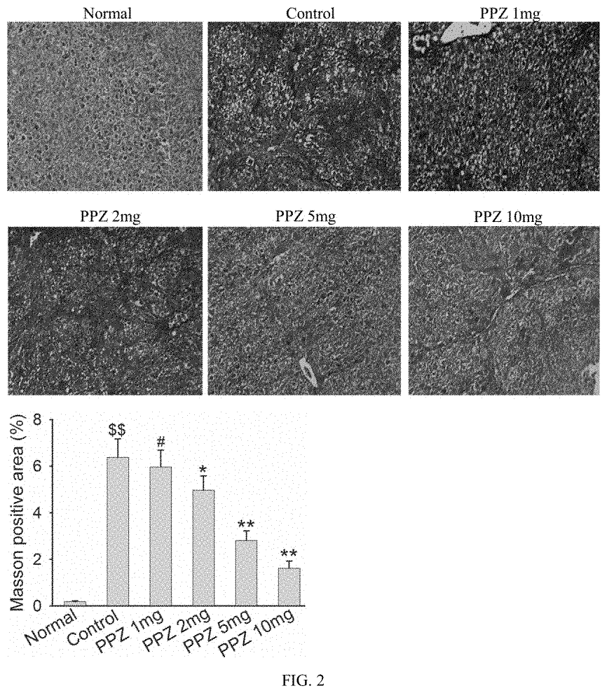

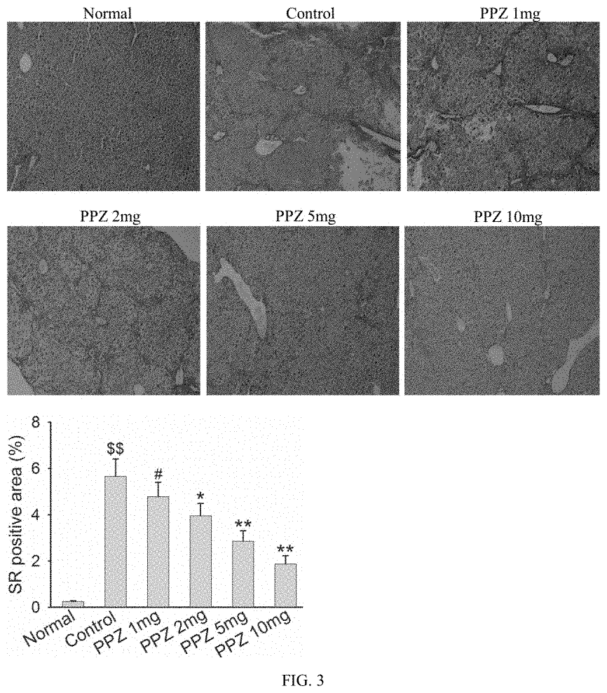

Masson and Sirius Red staining was mainly used to detect collagen fibers in tissues, which is an important indicator for tissue fibrosis. Therefore, the deposition of collagen fibers in liver tissue was detected by Masson and Sirius Red staining. The results showed that, compared with normal mice, mice administered with carbon tetrachloride had significantly-increased collagen fibers in liver tissue, and PPZ inhibited the increase of collagen fibers in liver tissue in a dosage-dependent manner (FIG. 2 and FIG. 3).

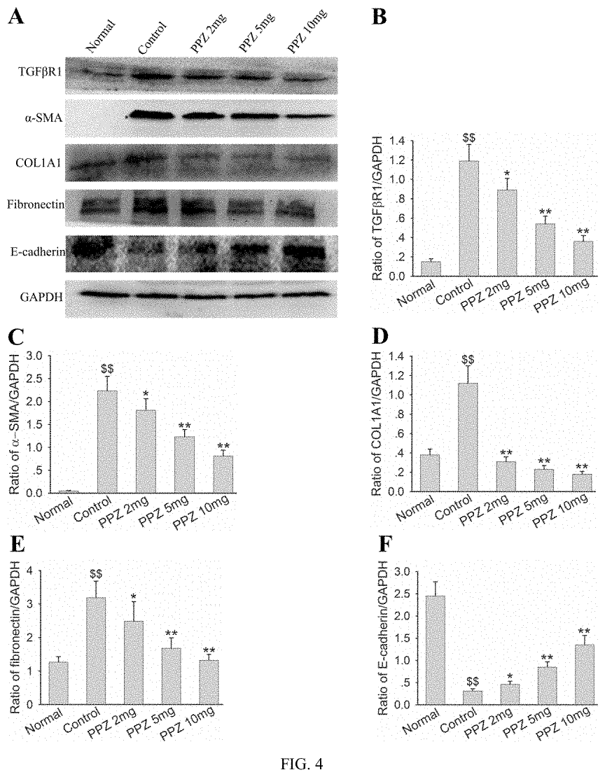

Hepatic stellate cells are the main source for myofibroblasts and play an important role in the occurrence of liver fibrosis. Profibrotic cytokines cause the occurrence of liver fibrosis by activating hepatic stellate cells to produce excessive extracellular matrix. Transforming growth factor β (TGFβ) is an important profibrotic cytokine. α-SMA, type I collagen (COL1) and fibronectin are main extracellular matrix proteins, and are important molecular markers for liver fibrosis. E-cadherin is an important epithelial phenotype marker for epithelial-mesenchymal transition. The effect of PPZ on these molecular markers in liver tissue was further examined in models of liver fibrosis induced by carbon tetrachloride. The expression levels of these molecular proteins were analyzed by Western Blot. The results showed that, compared with normal mice, mice administered with carbon tetrachloride exhibited significantly-increased expression of receptor TGFβR1 for cytokine TGFβ in liver tissue, remarkably-increased expression of extracellular matrix proteins α-SMA, COL1A1 and fibronectin, and significantly-reduced expression of epithelial phenotype protein E-cadherin, indicating that PPZ inhibited the expression of TGFβR1, α-SMA, COL1A1 and fibronectin in a dosage-dependent manner, and increased the expression of E-cadherin (FIG. 4).

The results showed that PPZ (proton pump inhibitor) inhibited the expression of receptor TGFβR1 for the profibrotic cytokine TGFβ caused by carbon tetrachloride, the activation of hepatic stellate cells and the epithelial-mesenchymal transition in liver, thereby inhibiting the occurrence of liver fibrosis. It is feasible to prepare a medicament that can effectively prevent and treat liver fibrosis with PPZ (proton pump inhibitor).

### Example 2

Effect of PPZ (Proton Pump Inhibitor) on the Activation of Human Hepatic Stellate Cells and the Expression of Liver Fibrosis-Related Proteins

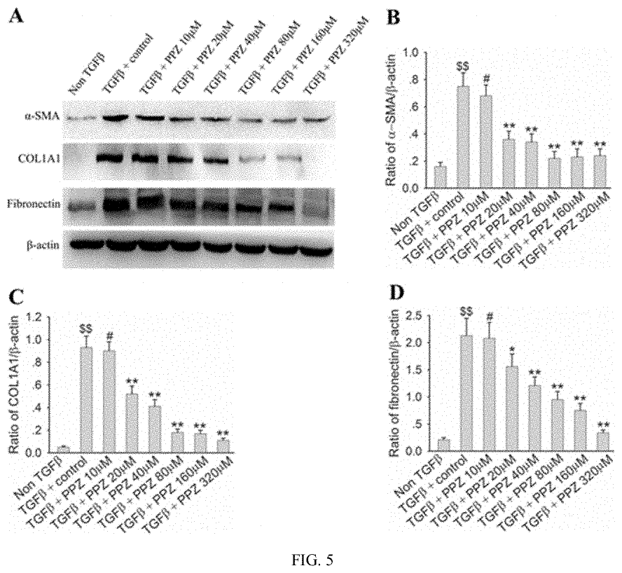

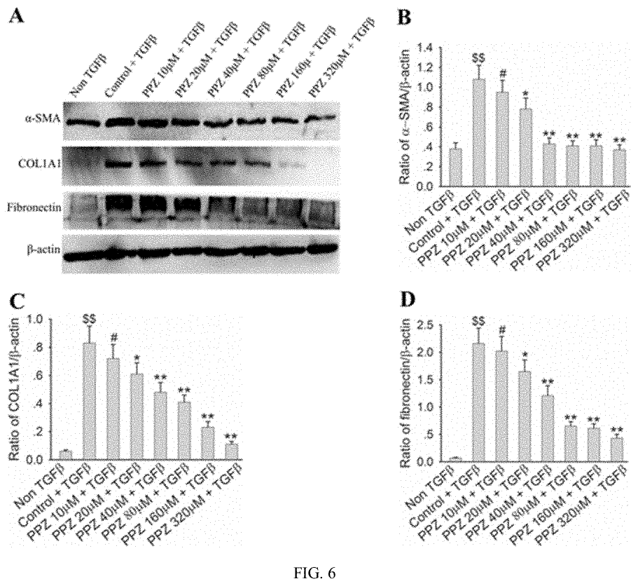

LX2 cells are human hepatic stellate cells. LX2 cells were adopted for experiment. First, LX2 cells were treated with TGFβ for 48 h, and then treated with 10 μM, 20 μM, 40 μM, 80 μM, 160 μM and 320 μM PPZ solutions and control solution (saline) separately for 24 h. It was observed whether TGFβ activated hepatic stellate cells, and the effect of PPZ on cells. The results showed that the expression of α-SMA, COL1A1 and fibronectin increased significantly after TGFβ treatment, and PPZ decreased the expression of these extracellular matrix proteins in a dosage-dependent manner (FIG. 5). For further experiments, LX2 cells were first pretreated with 10 μM, 20 μM, 40 μM, 80 μM, 160 μM and 320 μM PPZ solutions and control solution (saline) separately for 30 min, and then treated with TGFβ for 24 h. The results showed that PPZ inhibited the increased expression of α-SMA, COL1A1 and fibronectin caused by TGFβ in a dosage-dependent manner (FIG. 6).

The results showed that PPZ (proton pump inhibitor) inhibited the TGFβ-induced activation of hepatic stellate cells and the expression of extracellular matrix proteins.

The above results demonstrate that PPZ (proton pump inhibitor) can effectively inhibit the activation of hepatic stellate cells and the expression of liver fibrosis-related proteins, alleviate the liver damage in mice caused by carbon tetrachloride, and suppress the deposition of collagen fibers in the mouse liver, the epithelial-mesenchymal transition in liver and the occurrence of liver fibrosis. It is feasible to prepare a medicament that can effectively prevent and treat liver fibrosis with PPZ (proton pump inhibitor).

The above descriptions are merely preferred implementations of the present invention. It should be noted that a person of ordinary skill in the art may further make several improvements and modifications without departing from the principle of the present invention, but such improvements and modifications should be deemed as falling within the protection scope of the present invention.

What is claimed is:

1. A method of prevention and treatment of liver fibrosis comprising administering pantoprazole to a patient.

2. The method according to claim 1, wherein the pantoprazole prevents and treats liver fibrosis by inhibiting the activation of hepatic stellate cells.

3. A method of prevention and treatment of liver damage comprising administering pantoprazole to a patient.

4. The method according to claim 1, wherein the pantoprazole prevents and treats liver fibrosis by inhibiting the increase of collagen fibers in liver tissue.

5. The method according to claim 1, wherein the pantoprazole prevents and treats liver fibrosis by inhibiting the expression of extracellular matrix proteins.

6. The method according to claim 5, wherein the extracellular matrix protein comprises one or more of alpha smooth muscle actin (α-SMA), COL1A1 protein and fibronectin.

7. The method according to claim 1, wherein the pantoprazole prevents and treats liver fibrosis by increasing the expression of epithelial phenotype proteins.

8. A method of prevention and treatment of liver fibrosis comprising administering a composition comprising pantoprazole to a patient.
